(19) United States
(12) Patent Application Publication
(10) Pub. No.: US 2023/0077260 A1
(43) Pub. Date: Mar. 9, 2023

(54) ELECTRONIC ATOMIZATION DEVICE AND ATOMIZER THEREOF

(71) Applicant: SHENZHEN SMOORE TECHNOLOGY LIMITED, Shenzhen (CN)

(72) Inventors: Guilin LEI, Shenzhen (CN); Ru JIANG, Shenzhen (CN)

(21) Appl. No.: 17/986,144

(22) Filed: Nov. 14, 2022

Related U.S. Application Data
(63) Continuation of application No. PCT/CN2020/090649, filed on May 15, 2020.

(51) Int. Cl. A24F 40/44; A24F 40/10; A24F 40/485

(52) U.S. Cl. CPC A24F 40/44; A24F 40/10; A24F 40/485; A61M 11/045

(57) ABSTRACT

A vaporizer includes: an air inlet; an air outlet; an airflow channel in communication with the air inlet and the air outlet, respectively, a vaporization core being arranged in the airflow channel; a first capillary liquid absorbing structure and a second capillary liquid absorbing structure, the first capillary liquid absorbing structure and the second capillary liquid absorbing structure being arranged in the airflow channel provided between the air inlet and the vaporization core, the first capillary liquid absorbing structure being arranged between the vaporization core and the second capillary liquid absorbing structure; and a gap between the first capillary liquid absorbing structure and the second capillary liquid absorbing structure so as to allow air entering from the air inlet to reach the vaporization core after flowing through the gap and the first capillary liquid absorbing structure sequentially.

### CROSS-REFERENCE TO PRIOR APPLICATION

This application is a continuation of International Patent Application No. PCT/CN2020/090649, filed on May 15, 2020. The entire disclosure is hereby incorporated by reference herein.

### FIELD

This application relates to the field of vaporization device technologies, and in particular, to an electronic vaporization device and a vaporizer thereof.

### BACKGROUND

At present, an air inlet of an electronic vaporization device such as an e-cigarette is generally provided at a bottom portion of a vaporization cavity, and external air enters the vaporization cavity from the air inlet and then reaches a suction nozzle from an air outlet channel after being mixed with an aerosol substrate vaporized in the vaporization cavity. However, the vaporized aerosol substrate is easily condensed in the electronic vaporization device to form droplets, and aerosol substrate droplets formed through condensation are easily leaked from the air inlet of the electronic vaporization device to cause liquid leakage. As a result, the existing electronic vaporization device has a relatively poor liquid leakage-proof effect.

### SUMMARY

In an embodiment, the present invention provides a vaporizer, comprising: an air inlet; an air outlet; an airflow channel in communication with the air inlet and the air outlet, respectively, a vaporization core being arranged in the airflow channel; a first capillary liquid absorbing structure and a second capillary liquid absorbing structure, the first capillary liquid absorbing structure and the second capillary liquid absorbing structure being arranged in the airflow channel provided between the air inlet and the vaporization core, the first capillary liquid absorbing structure being arranged between the vaporization core and the second capillary liquid absorbing structure; and a gap between the first capillary liquid absorbing structure and the second capillary liquid absorbing structure so as to allow air entering from the air inlet to reach the vaporization core after flowing through the gap and the first capillary liquid absorbing structure sequentially.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic structural diagram of an embodiment of a vaporizer according to this application;

FIG. 2 is a schematic cross-sectional structural view of the vaporizer shown in FIG. 1 in a direction A-A;

FIG. 3 is a schematic partial structural diagram of the vaporizer shown in FIG. 2;

FIG. 4 is a schematic structural diagram of an embodiment of a second carrier according to this application;

FIG. 5 is a schematic exploded structural view of an embodiment of a vaporization core, a first carrier, and a second carrier according to this application;

FIG. 6 is a schematic cross-sectional structural view of the vaporizer shown in FIG. 1 in a direction B-B; and

FIG. 7 is a schematic structural diagram of an embodiment of an electronic vaporization device according to this application.

### DETAILED DESCRIPTION

In an embodiment, the present invention provides an electronic vaporization device and a vaporizer thereof, to improve a liquid leakage-proof effect of the vaporizer.

In an embodiment, the present invention provides a vaporizer. The vaporizer includes an air inlet, an air outlet, and an airflow channel. The airflow channel is in communication with the air inlet and the air outlet respectively, and a vaporization core is arranged in the airflow channel. The vaporizer further includes: a first capillary liquid absorbing structure and a second capillary liquid absorbing structure, where the first capillary liquid absorbing structure and the second capillary liquid absorbing structure are arranged in the airflow channel provided between the air inlet and the vaporization core, and the first capillary liquid absorbing structure is arranged between the vaporization core and the second capillary liquid absorbing structure; and a gap, between the first capillary liquid absorbing structure and the second capillary liquid absorbing structure, for air entering from the air inlet to reach the vaporization core after flowing through the gap and the first capillary liquid absorbing structure sequentially.

In an embodiment of this application, a plurality of air inlets are provided, and air entering from the plurality of air inlets flows through the first capillary liquid absorbing structure after being mixed at the gap.

In an embodiment of this application, the first capillary liquid absorbing structure includes a plurality of capillary grooves, the extending directions of the plurality of capillary grooves are parallel to each other and the cross-sectional areas thereof are the same, and the flow rates and the flow directions of airflows flowing through the capillary grooves are the same.

In an embodiment of this application, a block member is arranged in the airflow channel, the block member forms blocking between the gap and the air inlet, to limit the gap from being in direct communication with the air inlet, and the gap is in communication with the air inlet through the second capillary liquid absorbing structure.

In an embodiment of this application, the block member includes a first block member and a second block member, the planes in which the first block member and the second block member are located are arranged at an angle and the first block member and the second block member cooperatively encircle to form the gap, and after the first block member and the second block member are docked with the second capillary liquid absorbing structure, the gap is docked with the second capillary liquid absorbing structure, and the gap is in communication with the air inlet through the second capillary liquid absorbing structure.

In an embodiment of this application, the airflow channel includes an air inlet channel and an intermediate channel that are in communication with each other, the air inlet channel is further in communication with the air inlet, the intermediate channel is further in communication with the air outlet, and the vaporization core, the first capillary liquid absorbing structure, and the second capillary liquid absorbing structure are arranged in the intermediate channel.

In an embodiment of this application, the second capillary liquid absorbing structure is arranged close to the end opening of the air inlet channel, a first dam and a second dam that are arranged around the periphery of the end opening of the air inlet channel are arranged in the intermediate channel, the end opening is in communication with the intermediate channel, the first dam is arranged close to the gap relative to the second dam and the first dam forms blocking between the gap and the air inlet channel, to limit the gap from being in direct communication with the air inlet channel, and the gap is in communication with the air inlet channel through the second capillary liquid absorbing structure, and the first dam and the second dam are further configured to isolate the air inlet channel from the second capillary liquid absorbing structure.

In an embodiment of this application, the height of the first dam is higher than the height of the second dam, and a vent opening is formed between the first dam and the second dam, and the air inlet channel is in communication with the second capillary liquid absorbing structure through the vent opening and is further in communication with the gap.

In an embodiment of this application, heights of the first dam and the second dam are higher than the height of the second capillary liquid absorbing structure.

In an embodiment of this application, the first dam, the second dam, and the side wall of the bottom portion of the intermediate channel cooperatively surround the end opening of the air inlet channel, and the end opening is in communication with the intermediate channel.

In an embodiment of this application, the orthographic projection of the end opening of the air inlet channel on a reference plane is located outside the orthographic projection of the gap on the reference plane, the end opening is in communication with the intermediate channel, and the reference plane is perpendicular to the direction, along which the first capillary liquid absorbing structure and the second capillary liquid absorbing structure are opposite to each other.

In an embodiment of this application, the air inlet channel includes at least two first sub-channels, adjacent first sub-channels are connected through a second sub-channel, and the extending direction of the first sub-channel is different from the extending direction of the second sub-channel.

In an embodiment of this application, two opposite air inlet channels are provided on the bottom portion of the intermediate channel, and the gap is provided opposite to the second capillary liquid absorbing structure between the two air inlet channels.

In an embodiment of this application, the airflow channel further includes an air outlet channel, and the intermediate channel is in communication with the air outlet through the air outlet channel.

In an embodiment of this application, the first capillary liquid absorbing structure and the second capillary liquid absorbing structure are capillary grooves.

In an embodiment of this application, the first capillary liquid absorbing structure extends along its direction opposite to the second capillary liquid absorbing structure, the second capillary liquid absorbing structure includes a first capillary groove and a second capillary groove, the first capillary groove and the second capillary groove are in communication with each other and extend along different directions, and the plane defined by the extending directions of the first capillary groove and the second capillary groove is perpendicular to the extending direction of the first capillary liquid absorbing structure.

In an embodiment of this application, the vaporizer further includes a first carrier and a second carrier, the first carrier is docked with the second carrier to form the airflow channel, the vaporization core and the first capillary liquid absorbing structure are arranged in the first carrier, and the second capillary liquid absorbing structure is arranged in the second carrier.

In an embodiment of this application, the vaporizer further includes a third capillary liquid absorbing structure, and the third capillary liquid absorbing structure is arranged on the part of the inner wall of the airflow channel that is close to the vaporization core.

In an embodiment of this application, widths of capillary grooves of the first capillary liquid absorbing structure, the second capillary liquid absorbing structure, and the third capillary liquid absorbing structure are less than 1 mm.

To resolve the foregoing technical problems, another technical solution adopted by this application is to provide an electronic vaporization device. The electronic vaporization device includes a main unit and a vaporizer, where the main unit is connected to the vaporizer, and the vaporizer includes an air inlet, an air outlet, and an airflow channel. The airflow channel is in communication with the air inlet and the air outlet respectively, and a vaporization core is arranged in the airflow channel. The vaporizer further includes: a first capillary liquid absorbing structure and a second capillary liquid absorbing structure, where the first capillary liquid absorbing structure and the second capillary liquid absorbing structure are arranged in the airflow channel provided between the air inlet and the vaporization core, and the first capillary liquid absorbing structure is arranged between the vaporization core and the second capillary liquid absorbing structure; and a gap, between the first capillary liquid absorbing structure and the second capillary liquid absorbing structure, for air entering from the air inlet to reach the vaporization core after flowing through the gap and the first capillary liquid absorbing structure sequentially.

Beneficial effects of this application are as follows: Different from the related art, this application provides an electronic vaporization device and a vaporizer thereof. In this vaporizer, after an amount of accumulated liquid absorbed by the first capillary liquid absorbing structure reaches a threshold, the accumulated liquid in the first capillary liquid absorbing structure further enters the second capillary liquid absorbing structure and is absorbed by the second capillary liquid absorbing structure. That is, the vaporizer of this application increases a liquid storage (namely, accumulated liquid storage) amount through the first capillary liquid absorbing structure and the second capillary liquid absorbing structure, so that a risk of accumulated liquid leakage can be reduced, and a liquid leakage-proof effect of the vaporizer can be further improved.

In addition, the vaporizer further includes a gap between the first capillary liquid absorbing structure and the second capillary liquid absorbing structure of this application, for air entering from the air inlet to reach the vaporization core after flowing through the gap and the first capillary liquid absorbing structure sequentially, and the air is allowed to be uniformly mixed in the gap and is uniformly distributed to the first capillary liquid absorbing structure. Besides, the first capillary liquid absorbing structure further plays a role of airflow rectifying, so that the flow rates and the flow directions of airflows flowing through the first capillary liquid absorbing structure are relatively consistent, and the airflows in the vaporizer can be optimized to better carry an aerosol substrate vaporized at the vaporization core to the air outlet, thereby better providing vapor of the carried aerosol substrate to a user and helping improve the user experience.

In addition, during inhalation by the user, the aerosol substrate absorbed in the first capillary liquid absorbing structure may return to the vaporization core under driving of the airflows to be vaporized again, thereby improving the utilization of the aerosol substrate of the vaporizer in this application.

To make the objectives, technical solutions, and advantages of this application clearer, the following clearly and completely describes the technical solutions in the embodiments of this application with reference to the embodiments of this application. Apparently, the described embodiments are some rather than all of the embodiments of this application. All other embodiments obtained by a person of ordinary skill in the art based on the embodiments of this application without creative efforts shall fall within the protection scope of this application. The following embodiments and features in the embodiments may be mutually combined in a case that no conflict occurs.

To resolve the technical problem that a liquid leakage-proof effect of an electronic vaporization device in the related art is relatively poor, an embodiment of this application provides a vaporizer. The vaporizer includes an air inlet, an air outlet, and an airflow channel. The airflow channel is in communication with the air inlet and the air outlet respectively, and a vaporization core is arranged in the airflow channel. The vaporizer further includes: a first capillary liquid absorbing structure and a second capillary liquid absorbing structure, where the first capillary liquid absorbing structure and the second capillary liquid absorbing structure are arranged in the airflow channel provided between the air inlet and the vaporization core, and the first capillary liquid absorbing structure is arranged between the vaporization core and the second capillary liquid absorbing structure; and a gap, between the first capillary liquid absorbing structure and the second capillary liquid absorbing structure, for air entering from the air inlet to reach the vaporization core after flowing through the gap and the first capillary liquid absorbing structure sequentially. Detailed descriptions are provided below.

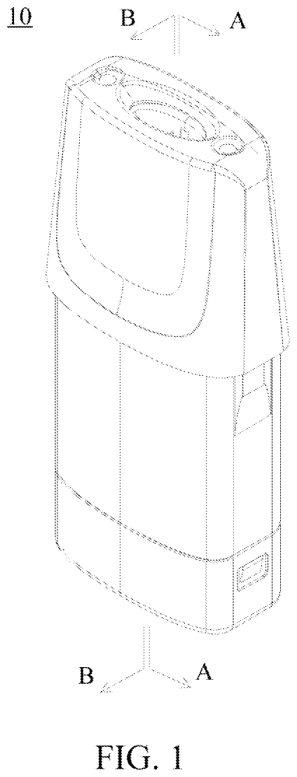

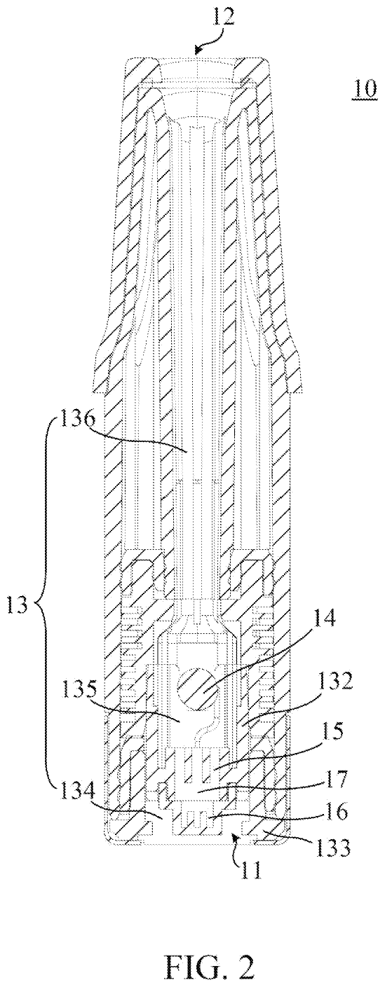

Referring to FIG. 1 and FIG. 2, FIG. 1 is a schematic structural diagram of an embodiment of a vaporizer according to this application, and FIG. 2 is a schematic cross-sectional structural view of the vaporizer shown in FIG. 1 in a direction A-A.

In an embodiment, the vaporizer 10 may be in a form such as an e-cigarette. Certainly, the vaporizer may also be a medical vaporization device applied to the medical field. A description is provided below by using the vaporizer 10 in a form of an e-cigarette as an example, which is not intended to limit this application.

Specifically, the vaporizer 10 includes an air inlet 11, an air outlet 12, and an airflow channel 13. The airflow channel 13 is in communication with the air inlet 11 and the air outlet 12 respectively, and a vaporization core 14 is arranged in the airflow channel 13, where the vaporization core 14 is configured to vaporize an aerosol substrate (for example, e-liquid or medical liquid) in the vaporizer 10.

A position of the air inlet 11 is a position where air enters the vaporizer 10. When a user performs inhalation, external air enters the airflow channel 13 from the air inlet 11, to carry the aerosol substrate vaporized by the vaporization core 14 in the airflow channel 13 to the air outlet 12, and output the aerosol substrate to the user along the air outlet 12 for inhalation by the user.

Optionally, the vaporization core 14 may be a porous heating body, which absorbs the aerosol substrate through capillary force and generates heat to vaporize the aerosol substrate. Preferably, the vaporization core 14 may be a porous ceramic heating body on which a heating film may be further arranged. Certainly, in some other embodiments of this application, the vaporization core 14 may also adopt a design of fiber cotton in cooperation with a heating wire, which is not limited herein.

The vaporizer 10 of this application further includes a first capillary liquid absorbing structure 15 and a second capillary liquid absorbing structure 16. The first capillary liquid absorbing structure 15 and the second capillary liquid absorbing structure 16 are arranged in the airflow channel 13 provided between the air inlet 11 and the vaporization core 14, and the first capillary liquid absorbing structure 15 is arranged between the vaporization core 14 and the second capillary liquid absorbing structure 16.

It should be noted that, because the first capillary liquid absorbing structure 15 is closer to the vaporization core 14 than the second capillary liquid absorbing structure 16, the aerosol substrate condensed in the airflow channel 13 may be first absorbed by the first capillary liquid absorbing structure 15. In addition, because the first capillary liquid absorbing structure 15 is in communication with the second capillary liquid absorbing structure 16, after an amount of accumulated liquid absorbed by the first capillary liquid absorbing structure 15 reaches a threshold, the accumulated liquid (namely, the aerosol substrate) in the first capillary liquid absorbing structure 15 further enters the second capillary liquid absorbing structure 16 and is absorbed by the second capillary liquid absorbing structure 16.

Specifically, when relatively few accumulated liquid exists in the airflow channel 13, the first capillary liquid absorbing structure 15 absorbs the aerosol substrate through capillary force to further lock the aerosol substrate. When relatively more accumulated liquid exists in the airflow channel 13, after the amount of accumulated liquid absorbed by the first capillary liquid absorbing structure 15 reaches a threshold, the aerosol substrate in the first capillary liquid absorbing structure 15 further enters the second capillary liquid absorbing structure 16 and is absorbed by the second capillary liquid absorbing structure 16 through capillary force.

That is, the vaporizer 10 of this application increases a liquid storage (namely, accumulated liquid storage, which is the same below) amount through the first capillary liquid absorbing structure 15 and the second capillary liquid absorbing structure 16, so that a risk of accumulated liquid leakage can be reduced, and a liquid leakage-proof effect of the vaporizer 10 can be further improved.

In addition, the vaporizer further includes a gap 17 between the first capillary liquid absorbing structure 15 and the second capillary liquid absorbing structure 16 in this embodiment. The air entering from the air inlet 11 reaches the vaporization core 14 after flowing through the gap 17 and the first capillary liquid absorbing structure 15 sequentially, the air is allowed to be uniformly mixed in the gap 17 and is uniformly distributed to the first capillary liquid absorbing structure 15, and the air further flows through the first capillary liquid absorbing structure 15 and carries the vaporized aerosol substrate to be outputted to the user. In addition, the first capillary liquid absorbing structure 15 further plays a role of airflow rectifying, so that the flow rates and the flow directions of airflows flowing through the first capillary liquid absorbing structure 15 are relatively consistent, the airflows can better cover the vaporization core 14, and the airflows in the vaporizer 10 can be optimized to better carry the aerosol substrate vaporized at the vaporization core 14 to the air outlet 12, thereby better providing vapor of the carried aerosol substrate to the user and helping improve the use experience of the user of the vaporizer 10.

In addition, during inhalation by the user, the aerosol substrate absorbed in the first capillary liquid absorbing structure 15 may return to the vaporization core 14 under driving of the airflows to be vaporized again, thereby improving the utilization of the aerosol substrate of the vaporizer 10 in this embodiment.

Optionally, the first capillary liquid absorbing structure 15 and the second capillary liquid absorbing structure 16 may be capillary grooves including capillary force, so that the aerosol substrate can be absorbed through capillary force. Certainly, the first capillary liquid absorbing structure 15 and the second capillary liquid absorbing structure 16 may also be other structures including capillary force, such as a structure including capillary force in a form of a frosting surface or lines formed by performing coarsening processing such as grinding on a surface of the airflow channel 13, that is, the first capillary liquid absorbing structure 15 and the second capillary liquid absorbing structure 16. Detailed descriptions are provided below.

In an embodiment, the first capillary liquid absorbing structure 15 includes a plurality of capillary grooves, the extending directions of the plurality of capillary grooves are parallel to each other and the cross-sectional areas thereof are the same, and the flow rates and the flow directions of airflows flowing through the capillary grooves are the same, thereby optimizing the airflow rectifying function of the first capillary liquid absorbing structure 15. Further, the plurality of capillary grooves may extend in a direction approaching the air outlet 12.

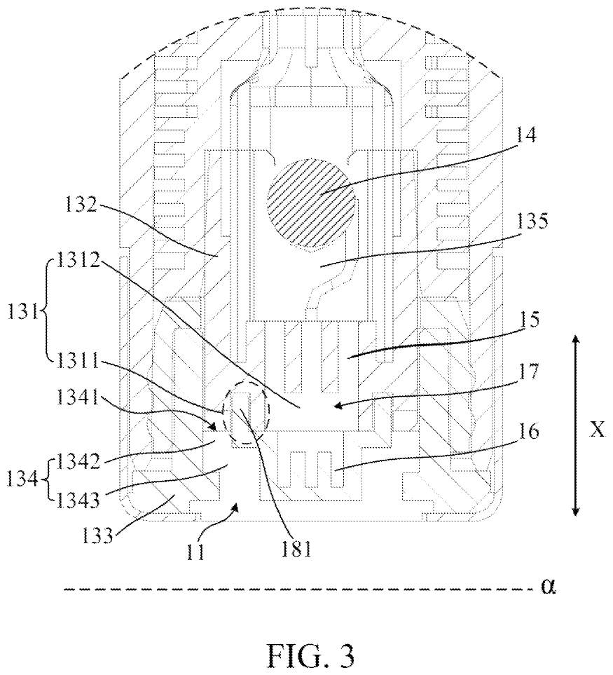

Referring to FIG. 2 and FIG. 3, FIG. 3 is a schematic partial structural diagram of the vaporizer shown in FIG. 2.

In an exemplary embodiment, the vaporizer 10 includes a plurality of air inlets 11. FIG. 2 and FIG. 3 show a situation that the vaporizer 10 includes two air inlets 11. After the air entering from the two air inlets 11 reaches the gap 17, the air can be mixed at the gap 17 and then reach the vaporization core 14 through the first capillary liquid absorbing structure 15. Specifically, the air entering from the two air inlets 11 is uniformly mixed at the gap 17 and is uniformly distributed to the first capillary liquid absorbing structure 15, and the air cooperates with the airflow rectifying function of the first capillary liquid absorbing structure 15 to optimize the airflows in the vaporizer 10 to better carry the aerosol substrate vaporized at the vaporization core 14 to the air outlet 12.

Certainly, in some other embodiments of this application, the vaporizer 10 may only include one air inlet 11, and the air entering from the air inlet 11 reaches the vaporization core 14 after flowing through the gap 17 and the first capillary liquid absorbing structure 15 sequentially, which is not limited herein.

Still referring to FIG. 2 and FIG. 3, in an embodiment, after the user stops inhalation, vapor in the vaporizer 10 may reflux. To prevent the refluxed vapor from directly leaking from the air inlet 11 and causing liquid leakage, a block member 131 is arranged in the airflow channel 13 in this embodiment, the block member 131 forms blocking between the gap 17 and the air inlet 11, to limit the gap 17 from being in direct communication with the air inlet 11, so that the gap 17 is in communication with the air inlet 11 through the second capillary liquid absorbing structure 16.

According to the foregoing method, the refluxed vapor first flows through the first capillary liquid absorbing structure 15, the condensed aerosol substrate in the vapor is first absorbed by the first capillary liquid absorbing structure 15, and the refluxed vapor reaching the gap 17 after flowing through the first capillary liquid absorbing structure 15 cannot directly escape from the air inlet 11 under limitation of the block member 131 but enters the second capillary liquid absorbing structure 16. Through secondary absorption by the second capillary liquid absorbing structure 16, most aerosol substrate in the refluxed vapor is locked in the vaporizer 10 and may not leak from the vaporizer 10. Arrangement of the block member 131 can reduce a risk that the refluxed vapor directly escapes from the air inlet 11, which further reduces a risk of liquid leakage and helps improve the liquid leakage-proof effect of the vaporizer 10.

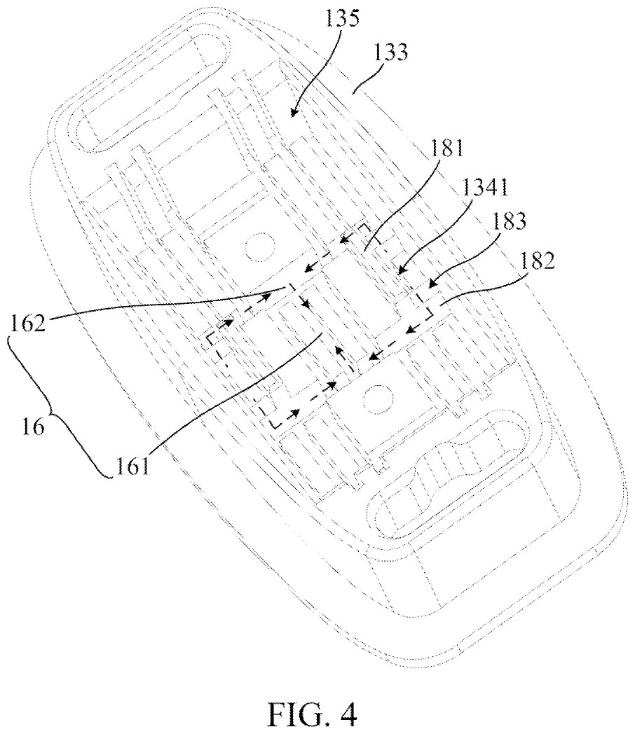

It should be noted that, the air inlet path of the vaporizer 10 in this embodiment is air inlet 11—second capillary liquid absorbing structure 16—gap 17—first capillary liquid absorbing structure 15—vaporization core 14—air outlet 12—user, and the air inlet path of the bottom portion of the airflow channel is shown by dashed arrows in FIG. 4. A vapor reflux path (after the user stops inhalation) of the vaporizer 10 in this embodiment is a reverse direction of the foregoing air inlet path, which specifically is air outlet 12—vaporization core 14—first capillary liquid absorbing structure 15—gap 17—second capillary liquid absorbing structure 16—air inlet 11. Due to a liquid absorbing function of the second capillary liquid absorbing structure 16 and limitation of the block member 131, the refluxed vapor can hardly escape from the air inlet 11, thereby greatly reducing the risk of liquid leakage.

Further, still referring to FIG. 2 and FIG. 3, the block member 131 includes a first block member 1311 and a second block member 1312, the planes in which the first block member 1311 and the second block member 1312 are located are arranged at an angle and the first block member and the second block member cooperatively encircle to form the gap 17, and after the first block member 1311 and the second block member 1312 are docked with the second capillary liquid absorbing structure 16, the gap 17 is docked with the second capillary liquid absorbing structure 16, so that the gap 17 is in communication with the air inlet 11 through the second capillary liquid absorbing structure 16. According to the foregoing method, under limitation of the first block member 1311 and the second block member 1312, the refluxed vapor can only directly enter the second capillary liquid absorbing structure 16 through the gap 17 and cannot directly escape from the air inlet 11.

Further, still referring to FIG. 2 to FIG. 4, the airflow channel 13 includes an air inlet channel 134 and an intermediate channel 135 that are in communication with each other, the air inlet channel 134 is further in communication with the air inlet 11, and the intermediate channel 135 is further in communication with the air outlet 12. The vaporization core 14, the first capillary liquid absorbing structure 15, and the second capillary liquid absorbing structure 16 are arranged in the intermediate channel 135.

Still further, the airflow channel 13 may further include an air outlet channel 136, and the intermediate channel 135 is in communication with the air outlet 12 through the air outlet channel 136.

The second capillary liquid absorbing structure 16 is arranged close to an end opening 1341 of the air inlet channel 134 in communication with the intermediate channel 135, and another end opening of the air inlet channel 134 opposite to the end opening 1341 is the air inlet 11. A first dam 181 and a second dam 182 that are arranged around a periphery of the end opening 1341 of the air inlet channel 134 in communication with the intermediate channel 135 are arranged in the intermediate channel 135. The first dam 181 is arranged close to the gap 17 relative to the second dam 182 and the first dam 181 forms blocking between the gap 17 and the air inlet channel 134, to limit the gap 17 from being in direct communication with the air inlet channel 134, so that the gap 17 is in communication with the air inlet channel 134 through the second capillary liquid absorbing structure 16. The first dam 181 may be a part of the first block member 1311 or the second block member 1312, FIG. 3 shows a situation that the first dam 181 is a part of the first block member 1311, and detailed descriptions are provided below.

Further, the height of the first dam 181 is higher than the height of the second dam 182 to form a vent opening 183 between the first dam 181 and the second dam 182. The air inlet channel 134 is in communication with the second capillary liquid absorbing structure 16 through the vent opening 183 and is further in communication with the gap 17. As shown in FIG. 3 and FIG. 4, air entering from the air inlet channel 134 needs to flow through the vent opening 183 to enter the second capillary liquid absorbing structure 16 and then reach the gap 17. A path that the air enters from the end opening 1341 of the air inlet channel and flows through the vent opening 183 to enter the second capillary liquid absorbing structure 16 is shown by dashed arrows in FIG. 4, where the end opening 1341 is in communication with the intermediate channel.

FIG. 4 shows a situation that the first dam 181, the second dam 182, and the side wall of the bottom portion of the intermediate channel 135 cooperatively surround the end opening 1341 of the air inlet channel, where the end opening 1341 is in communication with the intermediate channel, and the second dam 182 is arranged on two sides of the first dam 181 respectively, namely, the vent opening 183 is provided on two sides of the first dam 181 respectively. That is, the air entering from the air inlet channel flows through the vent openings 183 on two sides of the first dam 181 to enter the second capillary liquid absorbing structure 16.

FIG. 3 and FIG. 4 further show a situation that two opposite air inlet channels 134 are provided on the bottom portion of the intermediate channel 135. The gap 17 is arranged opposite to the second capillary liquid absorbing structure 16 between the two air inlet channels 134, the air entering from the two air inlet channels 134 turns to enter the second capillary liquid absorbing structure 16 between the two air inlet channels 134 and together enters the gap 17 after convergence, and the air then reaches the vaporization core 14 through the first capillary liquid absorbing structure 15, to carry the vaporized aerosol substrate to be outputted to the user.

In addition, still referring to FIG. 4, the first dam 181 and the second dam 182 are further configured to isolate the air inlet channel from the second capillary liquid absorbing structure 16. Specifically, the end opening 1341 of the air inlet channel is isolated from the second capillary liquid absorbing structure 16, and the end opening 1341 is in communication with the intermediate channel. In this way, even if an amount of the aerosol substrate absorbed by the second capillary liquid absorbing structure 16 is relatively large, the aerosol substrate absorbed by the second capillary liquid absorbing structure 16 may not leak from the air inlet channel, thereby further reducing the risk of liquid leakage.

Further, the heights of the first dam 181 and the second dam 182 are higher than the height of the second capillary liquid absorbing structure 16, so that the risk of liquid leakage can be further reduced on the basis of the arrangement of the first dam 181 and the second dam 182.

Still referring to FIG. 3, in an embodiment, the orthographic projection of the end opening 1341 of the air inlet channel 134 in communication with the intermediate channel 135 on a reference plane is located outside the orthographic projection of the gap 17 on the reference plane, and the reference plane (as shown by a plane a in FIG. 3) is perpendicular to the direction, along which the first capillary liquid absorbing structure 15 and the second capillary liquid absorbing structure 16 are opposite to each other (the direction along which the first capillary liquid absorbing structure 15 and the second capillary liquid absorbing structure 16 are opposite to each other is shown by an arrow X in FIG. 3). That is, for a direct-liquid vaporizer, the reference plane is perpendicular to a central axis of the vaporizer.

That is, the air inlet channel 134 and the gap 17 in this embodiment are provided in a misaligned manner on the reference plane, to prevent vapor refluxed from the gap 17 from directly escaping from the air inlet channel 134 and causing liquid leakage, which further reduces the risk of liquid leakage and helps improve the liquid leakage-proof effect of the vaporizer 10.

The orthographic projections of the two air inlet channels 134 shown in FIG. 3 on the reference plane are located on two opposite sides of the orthographic projection of the gap 17 on the reference plane.

Still referring to FIG. 3, in an embodiment, the air inlet channel 134 includes at least two first sub-channels 1342, adjacent first sub-channels 1342 are connected through a second sub-channel 1343, and the extending direction of the first sub-channel 1342 is different from the extending direction of the second sub-channel 1343. That is, the air inlet channel 134 extends zigzag, and the zigzag extended air inlet channel 134 increases the difficulty that the refluxed vapor escape from the air inlet channel 134, thereby further reducing the risk of liquid leakage and helping improve the liquid leakage-proof effect of the vaporizer.

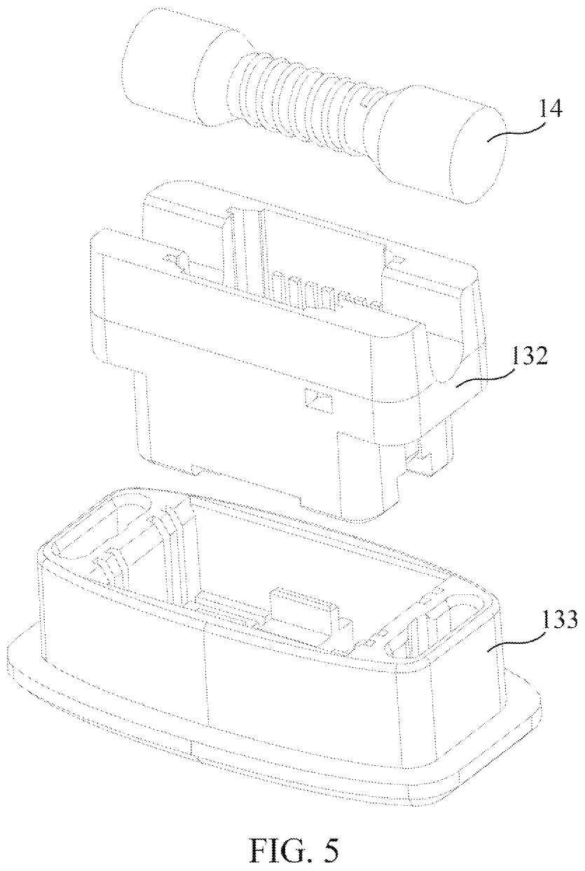

Referring to FIG. 2, FIG. 3, and FIG. 5, FIG. 5 is a schematic exploded structural view of an embodiment of a vaporization core, a first carrier, and a second carrier according to this application.

In an embodiment, the vaporizer 10 further includes a first carrier 132 and a second carrier 133. The first carrier 132 is docked with the second carrier 133 to form the airflow channel 13, and a cavity formed through docking between the first carrier 132 and the second carrier 133 is provided for flowing of air. The vaporization core 14 and the first capillary liquid absorbing structure 15 are arranged in the first carrier 132 and the first carrier 132 is in communication with the air outlet 12. The air inlet 11 and the second capillary liquid absorbing structure 16 are arranged in the second carrier 133.

That is, the airflow channel 13 of the vaporizer 10 in this embodiment adopts a separated structure design, which is beneficial to injection molding of various components. Specifically, the aerosol substrate leaked from the vaporization core 14 on the first carrier 132 is first absorbed by the first capillary liquid absorbing structure 15 on the first carrier 132. After an amount of accumulated liquid absorbed by the first capillary liquid absorbing structure 15 reaches a threshold, the aerosol substrate in the first capillary liquid absorbing structure 15 further infiltrates into the second capillary liquid absorbing structure 16. Because the second capillary liquid absorbing structure 16 on the second carrier 133 includes a relatively large liquid storage space, most infiltrated aerosol substrate can be absorbed, to prevent the aerosol substrate from leaking to the outside of the vaporizer 10.

Specifically, in the foregoing embodiment, the first dam 181 is arranged in the second carrier 133, a part of the first carrier 132 abutting against the second carrier 133 and the first dam 181 jointly form the first block member 1311 in the foregoing embodiment, and as shown in FIG. 3, the second block member 1312 in the foregoing embodiment is also arranged in the first carrier 132.

Certainly, in some other embodiments of this application, the first carrier 132 and the second carrier 133 may also be integrally formed through 3D printing, which is not limited herein.

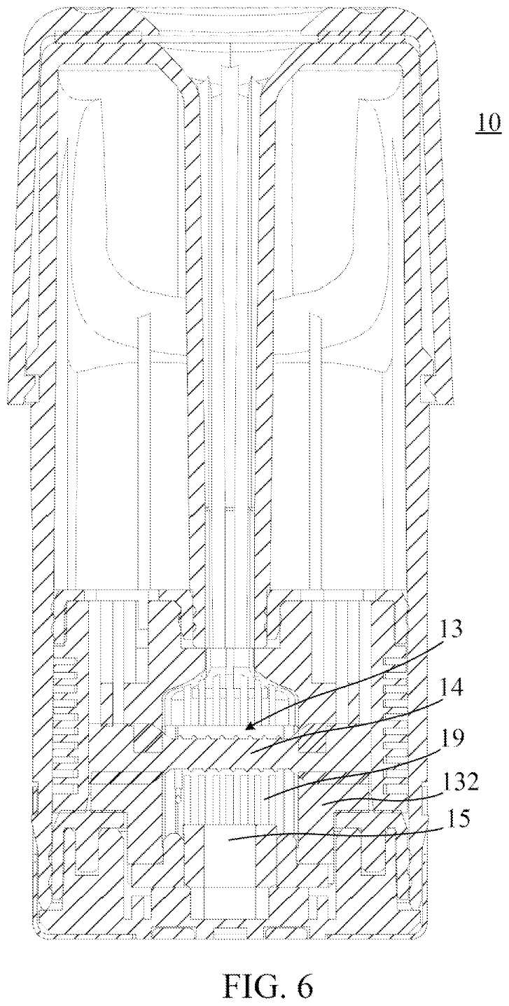

Referring to FIG. 6, FIG. 6 is a schematic cross-sectional structural view of the vaporizer shown in FIG. 1 in a direction B-B.

In an embodiment, the vaporizer 10 further includes a third capillary liquid absorbing structure 19. The third capillary liquid absorbing structure 19 is arranged on the part of the inner wall of the airflow channel 13 that is close to the vaporization core 14, and the third capillary liquid absorbing structure 19 is configured to cooperate with the first capillary liquid absorbing structure 15 to absorb accumulated liquid, so that a liquid storage amount in the vaporizer 10 is further increased, and the risk of liquid leakage is further reduced, which helps improve the liquid leakage-proof effect of the vaporizer 10.

Further, the third capillary liquid absorbing structure 19 is arranged in the first carrier 132 in the foregoing embodiment. Specifically, the third capillary liquid absorbing structure 19 is arranged on the side wall of the inner cavity of the first carrier 132, and the first capillary liquid absorbing structure 15 is arranged at the bottom portion of the inner cavity of the first carrier 132. The aerosol substrate absorbed by the third capillary liquid absorbing structure 19 may further flow toward the first capillary liquid absorbing structure 15 and is absorbed by the first capillary liquid absorbing structure 15.

It should be noted that, the first capillary liquid absorbing structure, the second capillary liquid absorbing structure, and the third capillary liquid absorbing structure may all be capillary grooves. As shown in FIG. 3, preferably, the first capillary liquid absorbing structure 15 extends along its direction opposite to the second capillary liquid absorbing structure 16. That is, the first capillary liquid absorbing structure 15 extends in a longitudinal direction. Certainly, the third capillary liquid absorbing structure may also extend in a longitudinal direction, which is not limited herein.

Still referring to FIG. 4, the second capillary liquid absorbing structure 16 includes a first capillary groove 161 and a second capillary groove 162. The first capillary groove 161 and the second capillary groove 162 are in communication with each other and extend along different directions. According to the foregoing method, a speed at which the second capillary liquid absorbing structure 16 absorbs the aerosol substrate is increased, and an effect that the second capillary liquid absorbing structure 16 absorbs the aerosol substrate is improved, thereby helping further reduce the risk of liquid leakage and improve the liquid leakage-proof effect of the vaporizer.

In this embodiment, for a direct-liquid vaporizer, the plane defined by the extending directions of the first capillary groove 161 and the second capillary groove 162 is perpendicular to the central axis of the vaporizer, and the capillary groove of the first capillary liquid absorbing structure extends in the direction of the central axis of the vaporizer.

Optionally, widths of capillary grooves of the first capillary liquid absorbing structure, the second capillary liquid absorbing structure, and the third capillary liquid absorbing structure are preferably less than 1 mm, so that the first capillary liquid absorbing structure, the second capillary liquid absorbing structure, and the third capillary liquid absorbing structure include a sufficient capillary liquid absorbing capability. If the width of the capillary groove is excessively large, the capillary liquid absorbing capability of the capillary groove is relatively weak and is not sufficient to meet a use requirement. In addition, a design value of the width of the capillary groove also depends on the viscosity of the aerosol substrate and the structure design limitation of the vaporizer. In addition, a greater depth of the capillary groove indicates a greater liquid storage amount. Therefore, in a case allowed by the structure, increasing the depth of the capillary groove helps increase the liquid storage amount of the capillary groove, thereby helping reduce the risk of liquid leakage.

Based on the above, according to the vaporizer provided in this application, after an amount of accumulated liquid absorbed by the first capillary liquid absorbing structure reaches a threshold, the accumulated liquid in the first capillary liquid absorbing structure further enters the second capillary liquid absorbing structure and is absorbed by the second capillary liquid absorbing structure. That is, the vaporizer of this application increases a liquid storage (namely, accumulated liquid storage) amount through the first capillary liquid absorbing structure and the second capillary liquid absorbing structure, so that a risk of accumulated liquid leakage can be reduced, and a liquid leakage-proof effect of the vaporizer can be further improved.

In addition, the vaporizer further includes a gap between the first capillary liquid absorbing structure and the second capillary liquid absorbing structure in this application. The air entering from the air inlet reaches the vaporization core after flowing through the gap and the first capillary liquid absorbing structure sequentially, the air is allowed to be uniformly mixed in the gap and is uniformly distributed to the first capillary liquid absorbing structure, and the air further flows through the first capillary liquid absorbing structure and carries the vaporized aerosol substrate to be outputted to the user. In addition, the first capillary liquid absorbing structure further plays a role of airflow rectifying, so that the flow rates and the flow directions of airflows flowing through the first capillary liquid absorbing structure are relatively consistent, the airflows can better cover the vaporization core, and the airflows in the vaporizer can be optimized to better carry the aerosol substrate vaporized at the vaporization core to the air outlet, thereby better providing vapor of the carried aerosol substrate to the user and helping improve the user experience.

Furthermore, a block member is arranged in the airflow channel of this application, and the block member forms blocking between the gap and the air inlet, to prevent the refluxed vapor from directly leaking from the air inlet and causing liquid leakage.

In addition, the air inlet channel and the gap of this application are provided in a misaligned manner. That is, an air inlet part and a main vaporization airway of the vaporizer of this application are provided in a misaligned manner, to prevent the vapor refluxed from the gap from directly escaping from the air inlet channel and causing liquid leakage.

In addition, during inhalation by the user, the aerosol substrate absorbed in the first capillary liquid absorbing structure may return to the vaporization core under driving of the airflows to be vaporized again, thereby improving the utilization of the aerosol substrate of the vaporizer of this application.

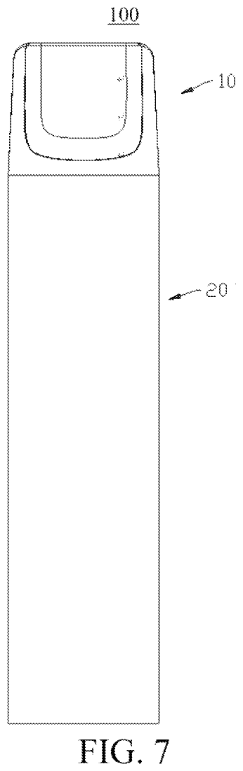

Referring to FIG. 7, FIG. 7 is a schematic structural diagram of an embodiment of an electronic vaporization device according to this application.

In an embodiment, the electronic vaporization device 100 includes a vaporizer 10 and a main unit 20. The vaporizer 10 is configured to heat and vaporize an aerosol substrate (for example, e-liquid). The main unit 20 is provided with a power supply and a control circuit. The vaporizer 10 may be fixedly connected to the main unit 20 or may be detachably connected to the main unit 20.

The vaporizer 10 includes an air inlet, an air outlet, and an airflow channel. The airflow channel is in communication with the air inlet and the air outlet respectively, and a vaporization core is arranged in the airflow channel. The vaporizer further includes: a first capillary liquid absorbing structure and a second capillary liquid absorbing structure, where the first capillary liquid absorbing structure and the second capillary liquid absorbing structure are arranged in the airflow channel provided between the air inlet and the vaporization core, and the first capillary liquid absorbing structure is arranged between the vaporization core and the second capillary liquid absorbing structure; and a gap, between the first capillary liquid absorbing structure and the second capillary liquid absorbing structure, for air entering from the air inlet to reach the vaporization core after flowing through the gap and the first capillary liquid absorbing structure sequentially. The vaporizer 10 has been described in detail in the foregoing embodiments, and details are not described herein again.

In addition, in this application, unless otherwise explicitly specified or defined, the terms such as “connect”, “connection”, and “stack” should be understood in a broad sense. For example, the connection may be a fixed connection, a detachable connection, or an integral connection; or a direct connection, an indirect connection through an intermediate, or internal communication between two elements or an interaction relationship between two elements. A person of ordinary skill in the art may understand the specific meanings of the foregoing terms in this application according to specific situations.

Finally, it should be noted that the foregoing embodiments are merely used for describing the technical solutions of this application, but are not intended to limit this application. Although this application is described in detail with reference to the foregoing embodiments, a person of ordinary skill in the art should understand that, modifications may still be made to the technical solutions in the foregoing embodiments, or equivalent replacements may be made to some or all of the technical features; and these modifications or replacements will not cause the essence of corresponding technical solutions to depart from the scope of the technical solutions in the embodiments of this application.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A vaporizer, comprising:
an air inlet;
an air outlet;
an airflow channel in communication with the air inlet and the air outlet, respectively, a vaporization core being arranged in the airflow channel;
a first capillary liquid absorbing structure and a second capillary liquid absorbing structure, the first capillary liquid absorbing structure and the second capillary liquid absorbing structure being arranged in the airflow channel provided between the air inlet and the vaporization core, the first capillary liquid absorbing structure being arranged between the vaporization core and the second capillary liquid absorbing structure; and
a gap between the first capillary liquid absorbing structure and the second capillary liquid absorbing structure so as to allow air entering from the air inlet to reach the vaporization core after flowing through the gap and the first capillary liquid absorbing structure sequentially.

2. The vaporizer of claim 1, wherein a plurality of air inlets are provided, and air entering from the plurality of air inlets flows through the first capillary liquid absorbing structure after being mixed at the gap.

3. The vaporizer of claim 1, wherein the first capillary liquid absorbing structure comprises a plurality of capillary grooves, extending directions of the plurality of capillary grooves being parallel to each other and cross-sectional areas thereof being the same, and
wherein flow rates and flow directions of airflows flowing through the capillary grooves are the same.

4. The vaporizer of claim 1, wherein a block member is arranged in the airflow channel, the block member providing blocking between the gap and the air inlet so as to limit the gap from being in direct communication with the air inlet, and
wherein the gap is in communication with the air inlet through the second capillary liquid absorbing structure.

5. The vaporizer of claim 4, wherein the block member comprises a first block member and a second block member, planes in which the first block member and the second block member are located being arranged at an angle and the first block member and the second block member cooperatively encircle to form the gap,
wherein, after the first block member and the second block member are docked with the second capillary liquid absorbing structure, the gap is docked with the second capillary liquid absorbing structure, and
wherein the gap is in communication with the air inlet through the second capillary liquid absorbing structure.

6. The vaporizer of claim 1, wherein the airflow channel comprises an air inlet channel and an intermediate channel that are in communication with each other, the air inlet channel is in communication with the air inlet, the intermediate channel is in communication with the air outlet, and
wherein the vaporization core, the first capillary liquid absorbing structure, and the second capillary liquid absorbing structure are arranged in the intermediate channel.

7. The vaporizer of claim 6, wherein the second capillary liquid absorbing structure is arranged close to an end opening of the air inlet channel, a first dam and a second dam that are arranged around a periphery of the end opening of the air inlet channel being arranged in the intermediate channel, the end opening being in communication with the intermediate channel,
wherein the first dam is arranged close to the gap relative to the second dam and the first dam provides blocking between the gap and the air inlet channel so as to limit the gap from being in direct communication with the air inlet channel,
wherein the gap is in communication with the air inlet channel through the second capillary liquid absorbing structure, and
wherein the first dam and the second dam are further configured to isolate the air inlet channel from the second capillary liquid absorbing structure.

8. The vaporizer of claim 7, wherein a height of the first dam is higher than a height of the second dam,
wherein a vent opening is formed between the first dam and the second dam, and
wherein the air inlet channel is in communication with the second capillary liquid absorbing structure through the vent opening and is in communication with the gap.

9. The vaporizer of claim 7, wherein heights of the first dam and the second dam are higher than a height of the second capillary liquid absorbing structure.

10. The vaporizer of claim 7, wherein the first dam, the second dam, and a side wall of the bottom portion of the intermediate channel cooperatively surround the end opening of the air inlet channel, and
wherein the end opening is in communication with the intermediate channel.

11. The vaporizer of claim 6, wherein an orthographic projection of the end opening of the air inlet channel on a reference plane is located outside an orthographic projection of the gap on the reference plane,
wherein the end opening is in communication with the intermediate channel, and
wherein the reference plane is perpendicular to a direction along which the first capillary liquid absorbing structure and the second capillary liquid absorbing structure are opposite to each other.

12. The vaporizer of claim 6, wherein the air inlet channel comprises at least two first sub-channels,
wherein adjacent first sub-channels are connected through a second sub-channel, and
wherein an extending direction of the first sub-channel is different from an extending direction of the second sub-channel.

13. The vaporizer of claim 6, wherein two opposite air inlet channels are provided on a bottom portion of the intermediate channel, and
wherein the gap is provided opposite to the second capillary liquid absorbing structure between the two air inlet channels.

14. The vaporizer of claim 6, wherein the airflow channel further comprises an air outlet channel, and
wherein the intermediate channel is in communication with the air outlet through the air outlet channel.

15. The vaporizer of claim 1, wherein the first capillary liquid absorbing structure and the second capillary liquid absorbing structure comprise capillary grooves.

16. The vaporizer of claim 15, wherein the first capillary liquid absorbing structure extends along its direction opposite to the second capillary liquid absorbing structure,
wherein the second capillary liquid absorbing structure comprises a first capillary groove and a second capillary groove, the first capillary groove and the second capillary groove being in communication with each other and extend along different directions, and
wherein a plane defined by extending directions of the first capillary groove and the second capillary groove is perpendicular to an extending direction of the first capillary liquid absorbing structure.

17. The vaporizer of claim 1, further comprising:
a first carrier and a second carrier, the first carrier being docked with the second carrier to form the airflow channel,
wherein the vaporization core and the first capillary liquid absorbing structure are arranged in the first carrier, and
wherein the second capillary liquid absorbing structure is arranged in the second carrier.

18. The vaporizer of claim 1, further comprising:
a third capillary liquid absorbing structure,
wherein the third capillary liquid absorbing structure is arranged on a part of the inner wall of the airflow channel that is close to the vaporization core.

19. The vaporizer of claim 18, wherein widths of capillary grooves of the first capillary liquid absorbing structure, the second capillary liquid absorbing structure, and the third capillary liquid absorbing structure are less than 1 mm.

20. An electronic vaporization device, comprising:
a main unit; and
a vaporizer, comprising:
an air inlet;
an air outlet;
an airflow channel in communication with the air inlet and the air outlet respectively, a vaporization core being arranged in the airflow channel;
a first capillary liquid absorbing structure and a second capillary liquid absorbing structure, the first capillary liquid absorbing structure and the second capillary liquid absorbing structure being arranged in the airflow channel provided between the air inlet and the vaporization core, the first capillary liquid absorbing structure being arranged between the vaporization core and the second capillary liquid absorbing structure; and
a gap between the first capillary liquid absorbing structure and the second capillary liquid absorbing structure so as to allow air entering from the air inlet to reach the vaporization core after flowing through the gap and the first capillary liquid absorbing structure sequentially,
wherein the main unit is connected to the vaporizer.
